(19) United States
(12) Patent Application Publication
(10) Pub. No.: US 2026/0325462 A1
(43) Pub. Date: Oct. 22, 2026

(54) TRANSDERMAL DELIVERY OF PRAMIPEXOLE AND RASAGILINE

(71) Applicant: Chin-Chih CHIANG, West Covina, CA (US)

(72) Inventor: Chin-Chih CHIANG, West Covina, CA (US)

(21) Appl. No.: 19/730,310

(22) Filed: Jul. 2, 2026

Related U.S. Application Data
(63) Continuation-in-part of application No. 18/481,850, filed on Oct. 5, 2023.
(60) Provisional application No. 63/415,059, filed on Oct. 11, 2022.

(51) Int. Cl. A61K 9/70; A61K 31/135; A61K 31/428

(52) U.S. Cl. CPC A61K 9/7061; A61K 31/135; A61K 31/428

(57) ABSTRACT

A transdermal patch includes a backing film, a multi-layered adhesive matrix and a release liner. The adhesive matrix is a laminate of several polyisobutylene based adhesive matrix and contains two active pharmaceutical ingredients, pramipexole and rasagiline. The transdermal patch is to be applied on skins for 7 days to deliver pramipexole and rasagiline for the treatment of Parkinson's disease.

### CROSS-REFERENCE TO RELATED APPLICATIONS

This application is a continuation-in-part of U.S. patent application Ser. No. 18/481,850, filed on Oct. 5, 2023, which is incorporated herein by reference in its entirety.

### FIELD OF THE INVENTION

The present invention relates to a transdermal patch which is composed of a release liner, a multi-layered adhesive matrix and a backing film. The multi-layered adhesive matrix is a laminate of several polyisobutylene (hereinafter “PIB”) based adhesive matrix containing two active pharmaceutically active ingredients, pramipexole and rasagiline and other functional ingredients which regulate the release of active ingredients from the patch and the skin permeation rates.

The present continuation-in-part application is directed to improvements over the parent application, particularly relating to multilayer adhesive matrix architectures and release-control systems for regulating the permeation of rasagiline free base. Additional transdermal patch configurations, matrix compositions, and manufacturing processes are disclosed herein.

### BACKGROUND

Parkinson's disease is a progressive disorder that is caused by degeneration of nerve cells in the part of the brain called the substantia nigra, which controls movement. These nerve cells die or become impaired, losing the ability to produce an important chemical called dopamine. Studies have shown that symptoms of Parkinson's disease develop in patients with an 80 percent or greater loss of dopamine-producing cells in the substantia nigra. Normally, dopamine operates in a delicate balance with other neurotransmitters to help coordinate the millions of nerve and muscle cells involved in movement. Without enough dopamine, this balance is disrupted, resulting in tremor (trembling in the hands, arms, legs and jaw); rigidity (stiffness of the limbs); slowness of movement; and impaired balance and coordination—the hallmark symptoms of Parkinson's disease.

Pramipexole has the ability to stimulate dopamine receptors in the striatum of the brain in the same way as dopamine, allowing patients to control their movement and mitigate their Parkinson's symptoms. Pramipexole is commercially available as pramipexole hydrochloride which is formulated as tablet dosage form. Pramipexole hydrochloride has been indicated for the treatment of Parkinson's disease and restless legs syndrome.

Rasagiline is a selective, irreversible MAO-B inhibitor indicated for the treatment of idiopathic Parkinson's disease. An enzyme called monoamine oxidase (MAO) breaks down neurotransmitters, dopamine. MAO has two forms, MAO-A and MAO-B. MAO-B is generally believed to break down dopamine. Rasagiline prevents the breakdown of dopamine by irreversibly binding to MAO-B. Dopamine is therefore more available, somewhat compensating for the diminished quantities made in the brains of people with Parkinson's disease. Rasagiline is commercially available as rasagiline mesylate which is formulated as tablet dosage form. Rasagiline mesylate has been indicated for the treatment of idiopathic Parkinson's disease.

The combination of pramipexole and rasagiline aiming to demonstrate the potentially synergistic mechanisms is shown in Phase 2 data of a significant symptomatic efficacy of P2B001, an oral tablet, versus placebo, with a benign safety profile that was similar to placebo.

Because of the low molecular weight and its lipophilic property, rasagiline has a fast penetration speed in the patch colloid and will be released from the colloid quickly. The present invention provides a long-term release transdermal patch product including pramipexole and rasagiline, which can be in use of treating Parkinson's disease externally. In the parent application, transdermal patches were designed to incorporate pramipexole and rasagiline (the drugs) in an adhesive matrix. Several commercially available adhesives, such as polyacrylate adhesive (Duro-Tak solution, a trade name products of Henkel), silicone elastomer adhesives (Bio-PSA, a trade name product of Dow) and PIB adhesives (Oppanol, a trade name products of BASF) had been screened. In-vitro skin permeation studies using human cadaver skins as membrane were conducted to investigate if both drugs could be released from the drug matrix and penetrating through skins at controlled rates for a duration of 7 days. Initial skin permeation study results indicated that pramipexole could permeate through skins at a controlled linear delivery for 7 days. However, rasagiline was showing a fast delivery on the first day and followed by slowing down rapidly due to the depletion of drug in the matrix. A transdermal patch that can stably release rasagiline and pramipexole is needed.

US Patent Application Publication US 2013/0072884 A1 disclosed a transdermal patch comprising a lamination of two adhesive matrices, wherein one matrix contains the active pharmaceutical ingredient (API), rasagiline mesylate, and the other matrix functions as a conversion layer containing a weak base. In the disclosed system, rasagiline mesylate is converted into its free base form after being released from the drug-containing matrix, diffusing through the conversion layer, and prior to permeation through the skin membrane. However, rasagiline mesylate is highly water-soluble, whereas the pressure-sensitive adhesives and the non-aqueous organic solvents commonly used for preparing adhesive matrices are generally incompatible with highly water-soluble compounds. As a result, rasagiline mesylate may exhibit limited solubility or dispersibility in the adhesive solution, thereby potentially leading to content uniformity issues and non-uniform particle size distribution within the matrix. In addition, the particle size of undissolved materials may require strict control in order to avoid coating defects or non-uniform coating thickness caused by gritty particles during the coating process. Such manufacturing challenges may increase process complexity, reduce production consistency, and increase manufacturing costs.

Therefore, providing a transdermal patch that can steadily release rasagiline and pramipexole is this invention aims to solve.

### SUMMARY OF THE INVENTION

The present invention develops a transdermal patch capable of steadily delivering pramipexole and rasagiline over multiple days of transdermal administration.

A drug in its base form is typically incorporated in adhesives to form a drug in adhesive matrix which is sandwiched between a release liner and a backing film to construct a transdermal patch. Before use, the release liner needs to be peeled off to expose the adhesive matrix which can be attached to skin. Once in a close contact with skin, drugs on the matrix surface will be transferring to the skin surface due to the concentration gradient. Once in the skin, the drugs will further be moving to the inner tissue. After the drugs are beginning to transfer from the matrix to skins, the drug in the inner matrix will start diffusing to the site where the drug is depleted. If the skin absorption or permeation of the drug is faster than the drug's travel inside the matrix, a skin absorption versus time profile of having an initial high absorption and a subsequent slowing down will result. In such a case, a strategy by incorporating a controlling layer to adjust the drug travel inside the matrix is necessary and needs to be carefully designed.

Thus a strategy of designing a double layered adhesive matrix by laminating a controlling adhesive matrix in which a variety of functional ingredients are included with a drug containing adhesive matrix is proposed. Study results indicated that Acrylates/C10-30 Alkyl Acrylate Crosspolymer (Pemulen TR-2, a trade name product of Lubrizol) and colloidal silicon dioxide (hereinafter “CSD”) were the ingredients in the controlling adhesive matrix which are able to regulate the skin permeation of rasagiline and the PIB was appropriate to be used as the adhesive to carry the drugs.

The current invention is to develop a transdermal patch which is able to deliver pramipexole and rasagiline at a controlled linear delivery for a 7-days duration. Initially a single layer drugs in adhesive matrix or a double layered patch by laminating a reservoir drugs in adhesive matrix with a controlling adhesive matrix without any other additives were designed. According to preliminary skin permeation results, it was found that pramipexole was able to be permeating through skins at a controlled linear rate however rasagiline was providing a fast permeating initially and a slowing down afterwards. Thus, a variety of additive was screened and included in the controlling adhesive matrix targeting to find out which one was able to regulate the skin permeation of rasagiline.

The skin permeation results was showing that Pemulen TR-2, a tackifier, and CSD, a filler, in the controlling adhesive matrix could serve the need of slowing down the initial skin permeation and maintain the delivery for a long duration. In addition, polyisobutylene (PIB) was selected as the adhesive for forming the matrix, while Pemulen TR-2 and colloidal silicon dioxide served as functional additives for controlling skin permeation. PIB was further found to be compatible with rasagiline free base and suitable for preparing a uniform adhesive matrix during the coating process. During the formulation preparation, PIB adhesive was first dissolved in n-heptane to form a solution or an emulsion with drugs or other ingredients. After the preparation, N-heptane could easily be evaporated during the manufacturing process under a low drying temperature and time due to its intermolecular binding was by Van Der Waals force, a low energy force.

The adhesive and additives in the controlling adhesive matrix has been screened and adopted. During the progress of product evaluation, it was found that a multi-layered adhesive matrix is needed which includes a first Drug Matrix, a Controlling Matrix, Placebo Matrix and a second Drug Matrix of which, pramipexole is formulated in the first Drug Matrix, Pemulen TR-2 and colloidal silicone dioxide are incorporate in Controlling Matrix, Placebo Matrix is set up to prevent the potential interaction between the Controlling Matrix and the second Drug Matrix, and rasagiline is formulated in the second Drug Matrix.

In the current invention, rasagiline free base is incorporated directly into an adhesive matrix. A unique controlling adhesive matrix containing specially selected functional ingredients as disclosed herein, is designed to regulate the diffusion and skin permeation of rasagiline as it passes through the matrix layer. The mechanism for controlling drug release in the present invention is fundamentally different from that disclosed US 2013/0072884 A1, which relies on a conversion layer to transform rasagiline mesylate into rasagiline free base prior to transdermal delivery.

Unlike the system disclosed in US 2013/0072884 A1, the present invention does not rely on a conversion layer to convert a rasagiline salt into rasagiline free base. Instead, rasagiline free base is directly incorporated into a dedicated drug matrix, while a separate controlling matrix comprising Acrylates/C10-30 Alkyl Acrylate Crosspolymer (Pemulen TR-2) and/or CSD is configured to regulate drug diffusion and skin permeation. This multilayer matrix architecture provides a distinct mechanism for controlling the release and transdermal delivery of rasagiline and is capable of providing a more controlled permeation profile over an extended delivery period. In the present invention, PIB is chosen as the adhesive and n-heptane is used as the solvent. Rasagiline base is soluble in the n-heptane-polyisobutylene mixture. A smooth and uniform film is able to be coated on a sheet of polymer membrane to be coated on. However, rasagiline mesylate is not soluble in n-heptane-polyisobutylene mixture which may cause a non-uniform issue during the manufacturing process. In addition, the particle sizes of the non-soluble materials need to be extremely well controlled in order to not cause non-uniform coatings due to any gritty particles.

By directly formulating rasagiline free base and employing a dedicated controlling matrix to regulate drug transport, the present invention provides a distinct release-control strategy that is capable of reducing the initial burst release of rasagiline and maintaining a more controlled permeation profile over an extended delivery period. To the best of the Applicant's knowledge, the prior art does not disclose a transdermal patch employing rasagiline free base in combination with such a release-controlling matrix for sustained transdermal delivery at controlled rates.

Following the filing of the parent application, further research and development were conducted. As a result, additional transdermal patch formulations, manufacturing processes, and matrix compositions were developed and are disclosed herein. In certain embodiments, the disclosure provides multilayer transdermal patches comprising pramipexole and rasagiline. In certain embodiments, the adhesive matrix comprises a polyisobutylene-based adhesive system. In certain embodiments, the disclosed formulations provide improved drug permeation, stability, and manufacturability.

The present disclosure provides multilayer transdermal patch systems comprising rasagiline and methods for manufacturing the same. In certain embodiments, the disclosure addresses the tendency of rasagiline to evaporate during drying due to its low melting point and volatility.

In certain embodiments, the transdermal patch comprises a polyisobutylene-based adhesive matrix containing rasagiline. The adhesive matrix may include a combination of high molecular weight PIB and low molecular weight PIB. Rasagiline may be incorporated into the adhesive matrix through a solution-based process using a volatile organic solvent and a mineral oil carrier.

In certain embodiments, the manufacturing process comprises preparing an adhesive solution, incorporating rasagiline into the adhesive matrix, coating the resulting composition onto a release liner, drying the coated layer under controlled conditions, and laminating the dried film with a backing film to form a multilayer transdermal patch.

In certain embodiments, the disclosed formulations and manufacturing conditions reduce loss of rasagiline during drying and improve the uniformity and stability of the transdermal patch.

### BRIEF DESCRIPTION OF THE DRAWING

FIG. 1 is a schematic view of a single-layered transdermal patch (left), and a double-layered transdermal patch (right).

FIG. 2 is skin permeation profile of pramipexole (left) and rasagiline (right) of Examples 1~5. Example 1—Duro-Tak 387-2052 adhesive having a function group of —COOH. Example 2—Duro-Tak 387-2074 adhesive having function groups of —OH and —COOH. Example 3—Duro-Tak 87-4098 adhesive having no function group. Example 4—Duro-Tak 387-2052 adhesive and Duro-Tak 387-2510 adhesive having a function group of —OH at a ratio of 1:2 by weight. Example 5—Duro-Tak 387-2052 adhesive and Duro-Tak 387-2510 adhesive at a ratio of 2:1 by weight.

FIG. 3 is a skin permeation profile of pramipexole (left) and rasagiline (right): Example 6 is a double-layered adhesive matrix having pramipexole and rasagiline in Duro-Tak 387-2052 and Duro-Tak 387-2510 at 2 to 1 ratio by weight as a reservoir adhesive matrix and Duro-Tak 387-2052 and Duro-Tak 387-2510 at 2 to 1 ratio by weight as the controlling adhesive matrix. Example 7 is a double-layered adhesive matrix having pramipexole and rasagiline in Duro-Tak 387-2052 and Duro-Tak 387-2510 at 1 to 2 ratio by weight as a reservoir adhesive matrix and Duro-Tak 387-2052 and Duro-Tak 387-2510 at 1 to 2 ratio by weight as a controlling adhesive matrix.

FIG. 4 is a skin permeation profile of pramipexole (left) and rasagiline (right): Example 8 is a double-layered adhesive matrix having pramipexole and rasagiline in PSA4302 as a reservoir and PSA4302 as a controlling adhesive matrix. Example 9 is a double-layered adhesive matrix having pramipexole and rasagiline in PSA4602 as a reservoir adhesive matrix and PSA4602 as a controlling adhesive matrix.

FIG. 5 is a skin permeation profile of pramipexole (left) and rasagiline (right): Example 10-Pramipexole and rasagiline in PIB as a reservoir adhesive matrix and CSD in PIB as a controlling adhesive matrix. Example 11—Pramipexole and rasagiline in PIB as a reservoir adhesive matrix and Talc in PIB as a controlling adhesive matrix. Example 12—Pramipexole and rasagiline in PIB as a reservoir adhesive matrix and Mg stearate in PIB as a controlling adhesive matrix. Example 13-Pramipexole and rasagiline in PIB as a reservoir adhesive matrix and Carbomer 934P in PIB as a controlling adhesive matrix. Example 14—Pramipexole and rasagiline in PIB as a reservoir adhesive matrix and Pemulen TR-2 in PIB as a controlling adhesive matrix.

FIG. 6 is a skin permeation profile of pramipexole (left) and rasagiline (right): Example 15-Pramipexole and rasagiline in PIB as a reservoir adhesive matrix and Pemulen TR-2 in PIB as a controlling adhesive matrix. Example 16—Pramipexole and rasagiline in PIB as a reservoir adhesive matrix and Pemulen TR-2 and CSD in PIB as a controlling adhesive matrix.

FIG. 7 is a schematic view of a six-layered transdermal patch, including a release liner, a four-layered adhesive matrix and a backing film.

FIG. 8 is skin permeation profiles of pramipexole and rasagiline: Example 21, Example 22 and Example 23 are each having 10%, 12% and 15% of Pemulen TR-2, respectively, in the Controlling Matrix.

FIG. 9 is skin permeation profiles of pramipexole and rasagiline from Example 24 and Example 25: Example 24, pramipexole and rasagiline are formulated in the second Drug Matrix. Example 25, pramipexole is formulated in the first Drug Matrix.

FIG. 10 is skin permeation profiles of pramipexole and rasagiline from Example 26 and Example 27: Both Examples are having the same compositions in each layer, however having a different coating thickness of the Controlling Matrix at 150 μm and 300 μm in Example 26 and Example 27, respectively.

FIG. 11 is skin permeation profiles of pramipexole and rasagiline from Example 28: Pramipexole and rasagiline are formulated in the first Drug Matrix and the second Drug Matrix, respectively.

### DETAILED DESCRIPTION OF THE INVENTION

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

The term “comprise” or “comprising” is generally used in the sense of include/including which means permitting the presence of one or more features, ingredients or components. The term “comprise” or “comprising” encompasses the term “consists” or “consisting of.”

As used herein, the term “treating” refers to the therapeutic measures to a disease or the symptoms or conditions of a disease, which include but are not limited to applying or administering one or more active agents to a subject suffering from the disease or the symptoms or conditions of the disease or exacerbation of the disease. The purpose of the therapeutic measures is to treat, cure, mitigate, relieve, alter, remedy, ameliorate, improve, or affect the disease, the symptoms or conditions of the disease, disability caused by the disease, or exacerbation of the disease. Specifically, the present invention provides pramipexole and rasagiline for treating Parkinson's disease.

As used herein, the term “effective amount” refers to the amount of an active ingredient achieving desired biological efficacy or therapeutic effects in a subject being treated.

The terms “about” and “approximately,” as used interchangeably herein, refer to within an acceptable error range for the particular value as determined by one of ordinary skill in the art, which will depend in part on how the value is measured or determined, i.e., the limitations of the measurement system. Alternatively, “about” can mean a range of up to up to 10%, up to 5%, up to 1%, up to 0.5%, or even up to 0.1% of a given value.

The current invention is related to a transdermal patch product. The patch product is composed of a release liner, an adhesive matrix, and a backing film. Both the release liner and backing film are used as a protective membrane. The adhesive matrix is a lamination of multi-layered adhesive matrix including a first Drug Matrix which is pramipexole in PIB matrix layer, Controlling Matrix which is containing a tackifier, a filler in PIB matrix layer, a Placebo Matrix separating the Controlling Matrix from a second Drug Matrix, and the second Drug Matrix which is containing rasagiline in PIB matrix layer. A plasticizer is used in all isobutylene matrixes. The first Drug Matrix is attached to the release liner which needs to be removed before the application of the adhesive matrix to the skin. The second Drug Matrix is adhered to the backing film. One patch product is to be applied on skin for 7 days to deliver pramipexole and rasagiline through skin for the treatment of Parkinson's disease.

Ideal drug candidates for the transdermal delivery must be low-molecular weight, lipophilic, low melting point and low daily oral dose. In addition, the transdermal delivery should be more attractive than oral delivery and more patient compliant if it shows a low oral bioavailability and needs frequent dosings daily. The current application is to describe a transdermal patch for a 7-day application to increase patient compliance.

In the current invention, the pharmaceutically active ingredients are pramipexole and rasagiline. The pharmaceutically inactive ingredients are PIB, an adhesive, a plasticizer, such as mineral oil, a tackifier, such as Pemulen TR-2, a trade name product of Lubrizol and a filler, such as colloidal silicone dioxide. The in vitro skin permeation studies using human cadaver skins were conducted to evaluate the amounts of drugs delivered through skins.

This invention is to design transdermal patch formulations which are composed of two pharmaceutically active ingredients-pramipexole and rasagiline, and other inactive ingredients such as filler, plasticizer, tackifier and carrier-adhesives and from which pramipexole and rasagiline can permeate through excised human cadaver skins in continuous and stable permeation profiles during 7-day permeation studies.

The transdermal patch is a multi-layered design, including release liner, adhesive matrix and backing layer. The adhesive matrix layer is either a single-layered or a double-layered design or a lamination of 4-layered design.

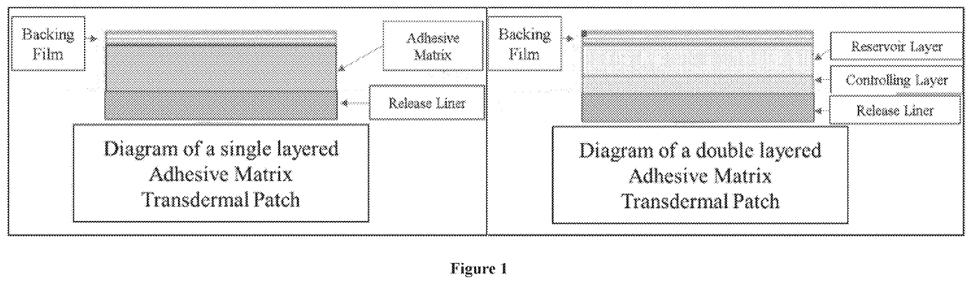

The single-layered matrix is composed of two pharmaceutically active ingredients and other inactive ingredients which are dissolved in adhesives. The picture of the transdermal patch in the current patent application is drawn as shown in FIG. 1.

The double layered matrix is a lamination of two single layered matrix including a reservoir layer and a controlling layer where one side of the reservoir layer is in contact with the backing layer while the other side is laminated with the controlling layer, and where one side of the controlling layer is in contact with the reservoir layer while the other side is in contact with the release liner.

The picture of the transdermal patch in the current patent application is drawn and shown in FIG. 1.

The multi-layered matrix is a lamination of four single layered matrix including a first Drug Matrix, a Controlling Matrix, a Placebo Matrix and a second Drug Matrix of which, the first Drug Matrix in attached to the release liner and the second Drug Matrix is adhered to the backing film.

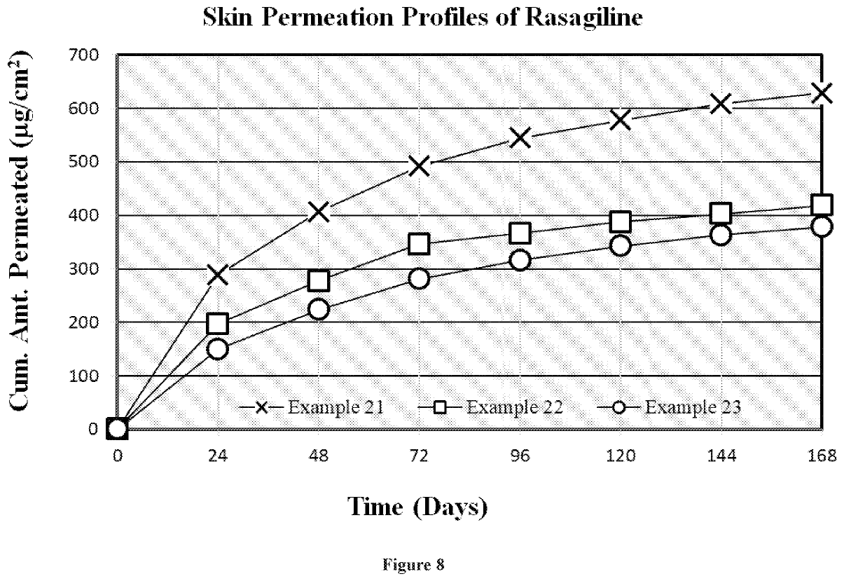

The picture of the transdermal patch in the current patent application is drawn and shown in FIG. 8.

The reservoir layer or Drug Matrix is enriched with pramipexole and rasagiline to provide sufficient drugs for a 7-day delivery and the controlling layer or Controlling Matrix is designed to control the release of both drugs from the patch and the delivery of drugs across the skins.

Before the application of skin patch, the release liner is removed to expose the adhesive matrix. Upon the application, the active ingredients start releasing from the matrix and are dissolved in the skin and then absorbed by the skin.

The release of active ingredients from the adhesive matrix is by a passive diffusion process. Once the patch is applied on the skin, a concentration gradient is created causing the active ingredients flow through the enriched layer to the less concentrated layer.

If the travel of active ingredients in the adhesive matrix is fast enough to supply those being depleted due to skin absorption during the targeted 7-day application, a single-layered matrix design is designed. If not, a double layered matrix is designed including a reservoir adhesive layer matrix to provide enough active ingredients and a controlling adhesive layer matrix to regulate the release of active ingredients.

A present invention is a transdermal patch formulation having pharmaceutically active ingredients of pramipexole and rasagiline and other inactive ingredients of a tackifier, a filler and a plasticizer in an adhesive, the carrier.

A further object of the present invention is the above formulation in which pramipexole is in its free base form and having its chemical name of(S)-2-amino-4,5,6,7-tetrahydro-6-(propylamino)benzothiazole, its empirical formula of C10H17N3S and a molecular weight of 211.3 and in which rasagiline is in its free base form and having its chemical name of (R)—N-(2-Propynyl)-2,3-dihydroinden-1-amine, its empirical formula of C12H13N and a molecular weight of 171.2.

In accordance of the present invention, the carrier is an adhesive in which two adhesive-matrix are formed, i.e., a reservoir layer containing drugs and a plasticizer in an adhesive which is designed to provide sufficient amounts of drugs, and a controlling layer containing a filler in an adhesive which is designed to regulate the drug release from the transdermal patch.

In accordance of the present invention, the adhesive in the above formulation is selected from the group consisting of polyacrylate adhesive, silicon adhesive and PIB adhesive.

In accordance of the present invention, the most preferred adhesive is PIB. PIB adhesives are marketed by BASF (sold under the names of Oppanol N-100, a high viscosity polymer having an average molecular weight of about 1,000,000 and Oppanol B-10, a low viscosity polymer having an average molecular weight of 10,000-50,000). A combination of Oppanol N-100 and Oppanol B-10 at a ratio of 1.0:1.5 to 1.5:1.0 by weight is preferred.

A further object of the present invention is the above formulation in which the filler is selected from the group consisting of CSD, acrylic acid polymers (sold under the name of Carbomer 934P), polyvinyl pyrrolidone K90, and Acrylates/C10-30 Alkyl Acrylate Crosspolymer (sold under the names of Pemulen TR-1, or Pemulen TR-2).

The most preferred filler is Pemulen TR-1 or Pemulen TR-2. Pemulen TR-2 is the trade name product of Lubrizol. Pemulen TR-2 is a polymeric emulsifier of a high molecular weight, crosslinked copolymer of acrylic acid and a hydrophobic C10-30 alkyl acrylate co-monomer. Its INCI Name is Acrylates/C10-30 Alkyl Acrylate Crosspolymer.

In accordance of the present invention, the amount of the filler is formulated in the range of 0.1% to 12% by weight.

A further object of the present invention is the above formulation in which a plasticizer is selected from the group consisting of mineral oil, light and mineral oil, heavy.

The most preferred plasticizer is mineral oil, light. In accordance of the present invention, the amount of the plasticizer is used in the range of 10% to 30% by weight.

A further object of the present invention is the above formulation in which the tackifier is selected from the group consisting of CSD, talc and magnesium stearate. A tackifier is used to adjust the integrity of the PIB based adhesive matrix.

The most preferred tackifier is CSD. In accordance of the present invention, the amount of the tackifier is formulated in the range of 3% to 15% by weight.

The transdermal patch delivers pramipexole at a skin permeation rate of 0.35-3.5 μg/cm² per hour.

The transdermal patch delivers rasagiline at a skin permeation rate of 0.2-3.6 μg/cm² per hour.

### EXAMPLES

### Examples 1-5 (Single-Layered Polyacrylate Adhesive Matrix)

The combination of pramipexole and rasagiline were formulated into polyacrylate adhesives, including those adhesives having different functional groups such as Duro-Tak 387-2052, Duro-Tak 87-2074, Duro-Tak 87-4908 and Duro-Tak 387-2516, which are having —OH/—COOH, none and —OH functional groups in their polyacrylate structures, respectively. Formulations of Example 1 to Example 5 are shown in the following table (Table 1). Patches which have a single-layered matrix are produced.

The formulations are pramipexole and rasagiline dissolved in Duro-Tak solutions. These solutions contain evaporable solvents, such as ethyl acetate and/or isopropyl alcohol which are removed during patch manufacturing process and the dried adhesive films are to be formed. Each dried film is designed to contain 8% pramipexole and 2% rasagiline.

Example 1—Duro-Tak 387-2052 adhesive having a function group of —COOH. Example 2—Duro-Tak 387-2074 adhesive having function groups of —OH and —COOH. Example 3—Duro-Tak 87-4098 adhesive having no function group. Example 4—Duro-Tak 387-2052 adhesive and Duro-Tak 387-2510 adhesive having a function group of —OH at a ratio of 1:2 by weight. Example 5—Duro-Tak 387-2052 adhesive and Duro-Tak 387-2510 adhesive at a ratio of 2:1 by weight.

TABLE 1
Formulations of Examples 1-5

| | | Example | | | | |
|---|---|---|---|---|---|---|
| | Function Groups of | 1 | 2 | 3 | 4 | 5 |
| Ingredients | Adhesives | Weight % | | | | |
| Pramipexole | — | 8 | 8 | 8 | 8 | 8 |
| Rasagiline | — | 2 | 2 | 2 | 2 | 2 |
| Lauroglycol* | — | 2 | 2 | 2 | 2 | 2 |
| Duro-Tak 387-2052 | —COOH | 88 | — | — | — | — |
| Duro-Tak 387-2074 | ′—OH/COOH | — | 88 | — | — | — |
| Duro-Tak 87-4098 | None | — | — | 88 | — | — |
| Duro-Tak 387-2052 & | —COOH and —OH | — | — | — | 88 | — |
| Duro-Tak 387-2510 | | | | | | |
| (1:2 by weight) | | | | | | |
| Duro-Tak 387-2052 & | —COOH and —OH | — | — | — | — | 88 |
| Duro-Tak 387-2510 | | | | | | |
| (2:1 by weight) | | | | | | |

Each ingredient is listed as the percentage weight.
*The chemical name of Lauroglycol is Propylene glycol monolaurate. It is a trade name sold by Gattefosse. It is commonly used as a skin permeation enhancer.

Manufacture of patches of Example 1 is described as in the following. Manufacture of other patches (Examples 2-5) follows the same process.

Add and dissolve 1.0 μm pramipexole and 0.25 μm rasagiline in a beaker containing 51.4 μm Duro-Tak 387-2052. Stir until homogeneous. Fix a sheet of release liner on the tray of Warner Mathis Coater. Pour approximately 5 μm of the drug containing adhesive solution on to the release liner. Use doctor knife to coat the adhesive solution evenly at a controlled thickness of 0.4 mm on the release liner. Send the tray into the drying oven which is preset at 60 °C for a drying time of 10 minutes. After drying, the tray is released out of the oven. Cover the dried drug containing adhesive film with a sheet of backing layer. Release the 3-layer sheet from the coating frame and cut the sheet into patches of desired sizes using a carver press die cutter.

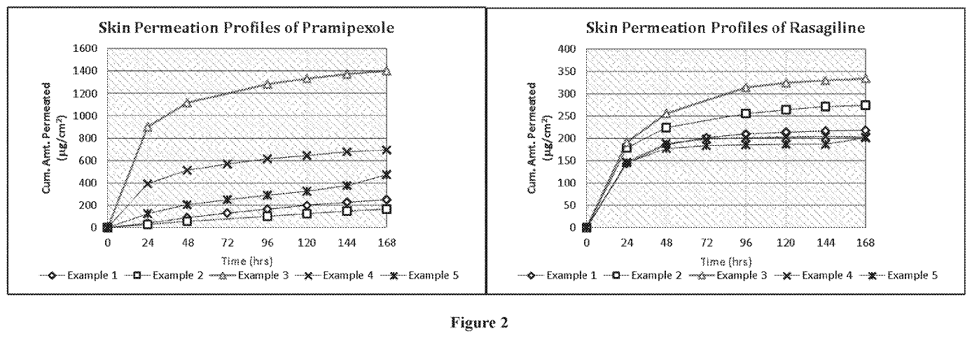

The patches thus made are subjected to the in-vitro skin permeation studies. A horizontal type skin permeation set up is used. Human cadaver skins are used as membrane having a calculated active permeation area of 0.64 cm². Each patch is applied on to the stratum corneum site of skin and the dermal site of skin is attached facing to each receptor compartment. In each receptor compartment, 3.5 mL of 20% polyethylene glycol 400 is added and used as the receptor medium. Samples, 0.1 mL each are taken with replacement every 24 hours to up to 168 hours. All samples were quantitated for pramipexole and rasagiline concentrations by HPLC. A typical HPLC chromatogram is shown in FIG. 2. Cumulated amount of pramipexole and rasagiline (in μg/cm²) versus time (in hours) profiles are plotted in FIG. 2 for Examples 1-5.

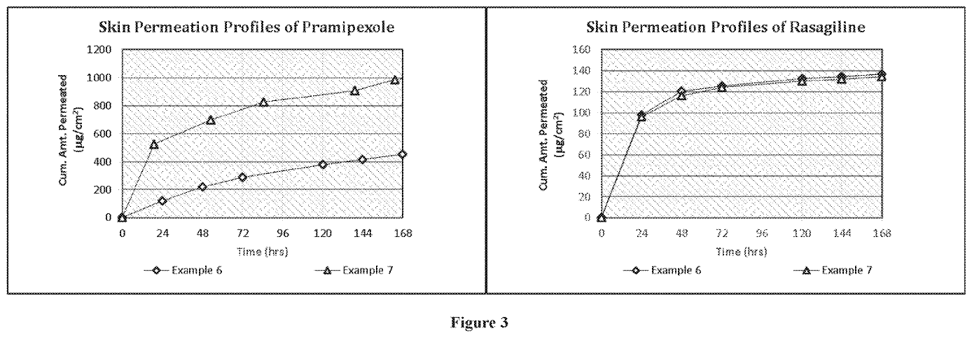

It is shown on FIG. 3 that pramipexole in Example 3 which is using the adhesive with no functional groups shows the highest skin permeation while that in the Example 1 which is using the adhesive with —COOH shows the lowest skin permeation profile. There is no continuous and stable absorption for Example 3, however, there is a 7-day continuous and stable absorption for Example 1. Surprisingly, as for rasagiline, all absorptions are showing non-linear profiles.

TABLE 2
The amount permeated across the skins from Example 3

| | Amt. permeated (μg/cm²/hr) | | | | | | |
|---|---|---|---|---|---|---|---|
| Example 1 | Day 1 | Day 2 | Day 3 | Day 4 | Day 5 | Day 6 | Day 7 |
| Pramipexole | 1.52 | 1.85 | 1.82 | 1.71 | 1.64 | 1.56 | 1.48 |
| Rasagiline | 5.99 | 3.87 | 2.80 | 2.18 | 1.78 | 1.50 | 1.30 |

Pramipexole: A constant delivery is averaged at 1.65 μg/cm²/hr during the 7-day delivery for pramipexole. However, it is not the case for rasagiline. The amount delivered is 5.99 μg/cm²/hr and decreases throughout other days.

### Examples 6-7 (Double-Layered Polyacrylate Adhesive Matrix)

Formulations of Example 6-7 are shown in the following table (Table 3). Example 6—Double-layered adhesive matrix having pramipexole and rasagiline in Duro-Tak 387-2052 and Duro-Tak 387-2510 at 2 to 1 ratio by weight as a reservoir adhesive matrix and Duro-Tak 387-2052 and Duro-Tak 387-2510 at 2 to 1 ratio by weight as the controlling adhesive matrix. Example 7—Double-layered adhesive matrix having pramipexole and rasagiline in Duro-Tak 387-2052 and Duro-Tak 387-2510 at 1 to 2 ratio by weight as a reservoir adhesive matrix and Duro-Tak 387-2052 and Duro-Tak 387-2510 at 1 to 2 ratio by weight as a controlling adhesive matrix.

TABLE 3
Formulations of Example 6-7

| | | Example | |
|---|---|---|---|
| | | 6 | 7 |
| Layer | Ingredients | Weight % | |
| Reservoir | Pramipexole | 8 | 8 |
| | Rasagiline | 1 | 1 |
| | Duro-Tak 387-2052 | 30 | 61 |
| | Duro-Tak 387-2510 | 61 | 30 |
| Controlling | Duro-Tak 387-2052 | 53.3 | 52 |
| | Duro-Tak 387-2510 | 26.7 | 26 |
| | PVP K90 | 20 | 20 |
| | SAIB | — | 2 |

Each ingredient is listed as the percentage weight.

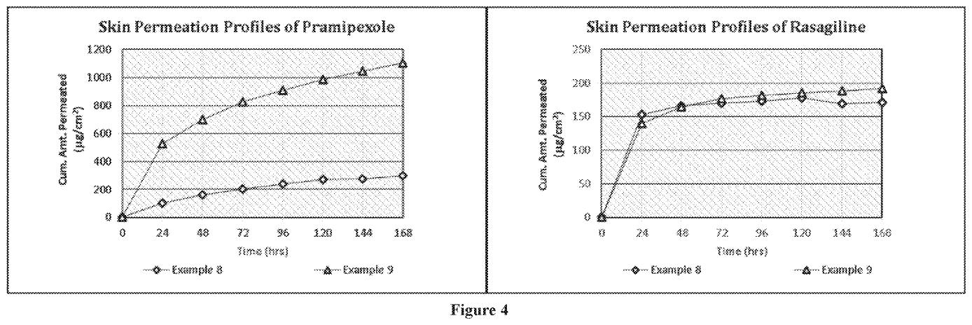

Skin permeation studies were performed. As shown in FIG. 4, the skin permeation of pramipexole slows down with the controlling layer having the polyacrylate adhesives and PVP only. Pramipexole shows a higher skin permeation when formulated in the adhesive with 2% SAIB (Sucrose Acetate Isobutyrate) added. For rasagiline, its skin permeation profiles show fast skin permeations on day 1 and slow down afterwards. In addition, the permeation is not different if additives in the controlling layer are different.

### Examples 8-9 (Double-Layered Silicone Adhesive Matrix)

Transdermal patches are formulated using pressure sensitive adhesives (PSA) as carriers. Two types of PSA, PSA-4302 and PSA-4602 which are trade name products of DOW are used. Table 4 shows formulations of Examples 8-9. Example 8 is a double-layered adhesive matrix having pramipexole and rasagiline in PSA4302 as a reservoir and PSA4302 as a controlling adhesive matrix. Example 9 is a double-layered adhesive matrix having pramipexole and rasagiline in PSA4602 as a reservoir adhesive matrix and PSA4602 as a controlling adhesive matrix. Manufacture of patches of Example 8 is described as in the following. Manufacture of patches of Example 9 follows the same process.

TABLE 4
Formulations of Example 8-9

| | | Example | |
|---|---|---|---|
| | | 8 | 9 |
| Layer | Ingredients | Weight % | |
| Reservoir | Pramipexole | 5 | 5 |
| | Rasagiline | 1 | 1 |
| | PSA4302 | 94 | — |
| | PSA4602 | — | 94 |
| Controlling | PSA4302 | 100 | — |
| | PSA4602 | — | 100 |

Each ingredient is listed as the percentage weight.

Manufacturing of the Reservoir Layer: Add and dissolve 1.54 μm pramipexole and 0.31 μm rasagiline in a beaker containing 48.16 μm PSA4302. Stir until homogeneous. Fix a sheet of backing layer on the tray of Warner Mathis Coater. Pour approximately 10 μm of the drug containing adhesive solution on to the backing layer. Use a doctor knife to coat the adhesive solution evenly at a controlled thickness of 0.4 mm on the backing layer. Send the tray into the drying oven which is preset at 60 °C for a drying time of 10 minutes. After drying, the tray is released out of the oven. Release the film and put it aside.

Manufacturing of the Controlling Layer: Fix a sheet of release liner on the tray of Warner Mathis Coater. Pour approximately 10 μm of PSA4302 on to the release liner. Use a doctor knife to coat PSA4302 solution evenly at a controlled thickness of 0.1 mm on the release liner. Send the tray into the drying oven which is preset at 60 °C for a drying time of 10 minutes. After drying, the tray is released out of the oven. Carefully laminate the adhesive side of Reservoir Layer on top the adhesive side of Controlling Layer avoiding air bubbles in between. Release the laminate sheet from the coating frame and cut the sheet into patches of desired sizes using a carver press die cutter.

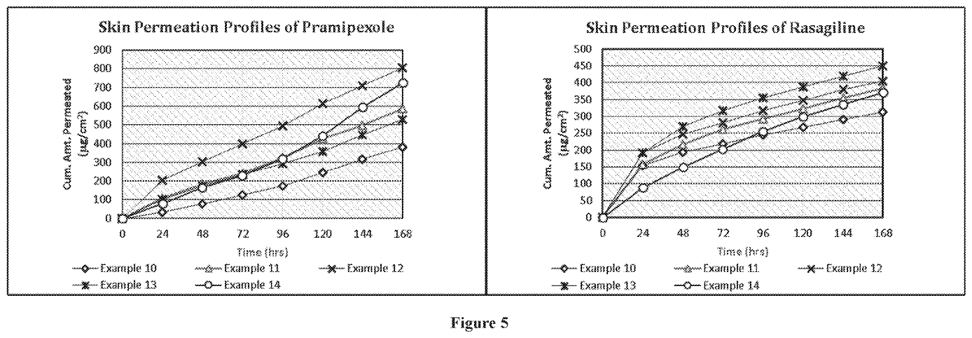

Skin permeation studies were performed. As shown in FIG. 5, pramipexole when formulated in PSA 4602 shows a higher skin permeation than it in PSA 4302. For rasagiline, its skin permeation is the same when formulated in either PSA 4302 or PSA 4602. However, non-linear skin permeations are resulted.

### Examples 10-14 (Double-Layered Polyisobutylene Adhesive Matrix)

Table 5 shows PIB based adhesive matrix formulations of Example 10-14. Oppanol, PIB adhesives, which is the trade name product of BASF is used. Mineral oil is a plasticizer for PIB adhesive. A variety of fillers is formulated in the PIB adhesive and they are used to investigate if they are able to regulate the delivery of rasagiline. As shown in Table 5, Examples 10-14 are having Pramipexole and rasagiline formulated in the reservoir layer and having fillers each formulated in the controlling layer. Manufacture of patches of Example 10 is described as in the following. Manufacture of patches of other formulations (Examples 10~14) follows the same process.

TABLE 5
Formulations of Example 10-14

| | | Example | | | | |
|---|---|---|---|---|---|---|
| Layer | Ingredients | 10 | 11 | 12 | 13 | 14 |
| Reservoir | Pramipexole | 5.75 | 5.75 | 5.75 | 5.75 | 5.75 |
| | Rasagiline | 1.44 | 1.44 | 1.44 | 1.44 | 1.44 |
| | Oppanol N-100 | 25.01 | 25.01 | 25.01 | 25.01 | 25.01 |
| | Oppanol B-10 | 31.26 | 31.26 | 31.26 | 31.26 | 31.26 |
| | Mineral Oil, Heavy | 36.55 | 36.55 | 36.55 | 36.55 | 36.55 |
| Controlling | Oppanol N-100 | 24.32 | 24.32 | 24.32 | 25.59 | 25.59 |
| | Oppanol B-10 | 30.4 | 30.4 | 30.4 | 31.99 | 31.99 |
| | Mineral Oil, Heavy | 35.54 | 35.54 | 35.54 | 37.4 | 37.4 |
| | CSD | 9.74 | — | — | — | — |
| | Talc | — | 9.74 | — | — | — |
| | Mg Stearate | — | — | 9.74 | — | — |
| | Carbomer 934P | — | — | — | 5.02 | — |
| | Pemulen TR-2 | — | — | — | — | 5.02 |

Each ingredient is listed as the percentage weight.

Manufacturing of the Reservoir Layer: Add and dissolve Oppanol N-100, Oppanol B-10 and Mineral Oil, heavy in n-heptane to form an adhesive solution. Add pramipexole and rasagiline into the adhesive solution. Homogenize the mixture. Fix a sheet of backing layer on the tray of Warner Mathis Coater. Pour approximately 10 μm of the drug containing adhesive solution on to the backing layer. Use a doctor knife to coat the adhesive solution evenly at a controlled thickness of 0.4 mm on the backing layer. Send the tray into the drying oven which is preset at 60 °C for a drying time of 10 minutes. After drying, the tray is released out of the oven. Release the film and put it aside.

Manufacturing of the Controlling Layer: Add and dissolve Oppanol N-100, Oppanol B-10 and Mineral Oil, heavy in n-heptane to form an adhesive solution. Homogenize Talc in n-heptane. Add the mixture into the adhesive solution and homogenize. Fix a sheet of release liner on the tray of Warner Mathis Coater. Pour approximately 10 μm of the mixture on to the release liner. Use a doctor knife to coat the mixture evenly at a controlled thickness of 0.1 mm on the release liner. Send the tray into the drying oven which is preset at 60 °C for a drying time of 10 minutes. After drying, the tray is released out of the oven. Carefully laminate the adhesive side of Reservoir Layer on the adhesive side of Controlling Layer avoiding air bubbles in between. Release the laminate sheet from the coating frame and cut the sheet into patches of desired sizes using a carver press die cutter.

FIG. 5 shows that pramipexole in Example 14 shows a controlled and stable skin delivery for a 7 day duration having an average hourly delivery of 3.62 μg/cm². Surprisingly, rasagiline is also showing a controlled and stable skin delivery for a 7 day duration having an average hourly delivery of 2.75 μg/cm². All other absorptions are showing non-linear profiles.

TABLE 6
The amount permeated across the skins from Example 14

| | Amount Permeated (μg/cm²/hr) | | | | | | |
|---|---|---|---|---|---|---|---|
| Example 14 | Day 1 | Day 2 | Day 3 | Day 4 | Day 5 | Day 6 | Day 7 |
| Pramipexole | 3.28 | 3.43 | 3.17 | 3.32 | 3.67 | 4.14 | 4.30 |
| Rasagiline | 3.68 | 3.11 | 2.81 | 2.66 | 2.49 | 2.33 | 2.20 |

### Examples 15-16 (Double-Layered Polyisobutylene Adhesive Matrix)

Table 7 shows formulations of Examples 15-16. Pramipexole and rasagiline are incorporated in the reservoir adhesive matrix. In Example 15, Pemulen TR-2 is formulated in the controlling adhesive matrix. In Example 16 both Pemulen TR-2 and CSD are formulated in the Controlling Matrix.

TABLE 7
Formulations of Example 15-16

| | | Example | |
|---|---|---|---|
| | | 15 | 16 |
| Layer | Ingredients | Weight % | |
| Controlling | CSD | — | 5.1 |
| | Pemulen TR-2 | 4.9 | 5.1 |
| | Mineral Oil | 37.4 | 35.3 |
| | Oppanol N-100 | 25.6 | 24.2 |
| | Oppanol B-10 | 32 | 30.2 |
| Reservoir | Pramipexole | 5.6 | 5.6 |
| | Rasagiline | 1.4 | 1.4 |
| | Mineral Oil | 36.6 | 36.6 |
| | Oppanol N-100 | 25.1 | 25.1 |
| | Oppanol B-10 | 31.3 | 31.3 |

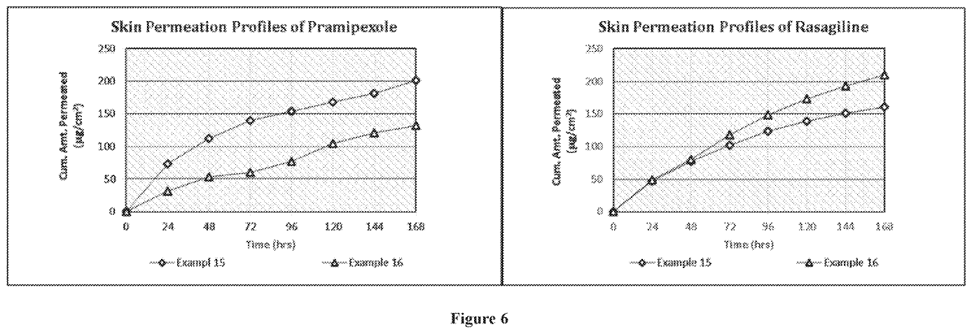

FIG. 6 shows the skin permeation profile of pramipexole and rasagiline. Example 15 with Pemulen TR-2 in the controlling layer is showing a faster delivery pramipexole than Example 16 which not only has Pemulen TR-2 also has CSD added. CSD seems not having much impact on the skin permeation of rasagiline. However, importantly CSD as a filler/reinforcement is reported to be able to increase the integrity, cohesive strength, and durability of PIB adhesives.

### Example 17 (Using Polyisobutylene to Make Adhesive Matrix)

PIB is used as a base to prepare adhesive matrix-PIB adhesive matrix. In the current application, a mixture of two commercially available PIB such as Oppanol N-100 and Oppanol B-10, both are trade name products of BASF, at a weight ratio of 1:1.25 are used. Oppanol N-100 is a high molecular weight polyisobutylene (HMW PIB) and Oppanol B-10 is a low molecular weight polyisobutylene (LMW PIB). At a weight ratio of 1:1.5, mineral oil, as a plasticizer, is added into the PIB mixture.

### Example 18 (Optimization of Manufacturing Process of Rasagiline in Polyisobutylene Adhesive)

PIB adhesive is used to form adhesive matrix in the current invention. It is critical to make sure if the active ingredients in PIB based adhesive solution will loss or not during the drying process. Pramipexole has a boiling point of 378 °C at 760 mmHg. It won't be evaporated during the commonly used drying temperatures of around 60-70 °C. However, rasagiline has a low boiling point at 35-41 °C. It may be evaporated along with n-heptane which is the solvent used to dissolve PIB adhesives and needs to be removed to form a dried adhesive matrix. The chemical structure of n-heptane is H₃C—(CH₂)₅—CH₃. Its boiling point is approximately 98.4 °C (208.9 °F) at standard atmospheric pressure. However, it can be evaporated at a temperature lower than its boiling point due to that its intermolecular interactions are due to van der Waals forces. It is critical to optimize the drying parameters such as drying temperatures and drying time to prevent rasagiline loss during the manufacturing process. The following provides the method.

A single layered PIB adhesive matrix containing 5% rasagiline in PIB adhesive matrix is prepared. The solution preparation and drying parameters to form the matrix are described in the following.
1. Preparation of PIB Solution: In the present invention, the adhesive is a combination of high molecular weight polyisobutylene (HMW PIB-Oppanol N-100) and low molecular weight polyisobutylene (LMW PIB-Oppanol B-10) at a ratio of 1:1.25 by weight. Both Oppanol N-100 and Oppanol B-10 are added into a container. N-Heptane is added into the mixture (3 times the weight of Oppanol N-100 and Oppanol B-10) and stirred to dissolve the adhesive.
2. Rasagiline in PIB Solution: Rasagiline is first mixed and dissolved in mineral oil at 40 °C. PIB solution is added into the mixture.
3. Coating, Drying and Lamination: The rasagiline in PIB solution is cast on a sheet of release liner with a coating thickness controlled at 400 μm which is dried in an oven to evaporate n-heptane at pre-determined drying temperature and drying time. The dried film is then laminated with a sheet of backing film to form a 3-layered laminate. Repeat the above process by applying different drying temperature and time to construct a serial patches.
4. Cutting: The laminates are cut into patches of 5 cm² each in size.
5. Extraction and Assay: After peeling off the release liner which is covered on the adhesive matrix of each patch, the remaining patch is dropped into a vial. Methanol is used as the solvent to extract rasagiline by vortexing the vials vigorously. The methanol extract is filtered. The filtrates are analyzed for rasagiline content. Table 8 shows the analytical results.

TABLE 8
Analytical results of Patches produced at
different Manufacturing Conditions

| Drying | | | Rasagiline | | |
|---|---|---|---|---|---|
| Conditions | Patch | Matrix | Assay | | |
| Temperature/ | Wt. | Wt* | (mg) By | By | % |
| Duration | (mg) | (mg) | Weight** | HPLC | Assay*** |
| 140 °F/10 min | 109.8 | 29.5 | 1.48 | 1.03 | 70% |
| 140 °F/5 min | 113.2 | 32.9 | 1.64 | 1.44 | 88% |
| 149 ºF/3 min | 112.1 | 31.8 | 1.59 | 1.42 | 89% |
| 122 ºF/5 min | 113.4 | 33.1 | 1.65 | 1.51 | 91% |
| 122 ºF/3 min | 112.1 | 31.8 | 1.59 | 1.58 | 100% |
| 113 ºF/5 min | 112.6 | 32.3 | 1.62 | 1.6 | 99% |
| 113 ºF/3 min | 113.7 | 33.4 | 1.67 | 1.64 | 98% |

*Matrix wt. = Patch wt.-80.3 (wt. of release liner plus wt. of backing film)
**Rasagiline Assay by Weight = Matrix Wt. × 0.05 (Each matrix contains 5% rasagiline)
***% Assay = Assay by HPLC/Assay by Wt.

The optimized drying conditions of rasagiline in PIB adhesive matrix is thus determined to be at 122 °F for 3 minutes.

### Examples 19 (a 6-Layered Matrix Design of a Transdermal Patch)

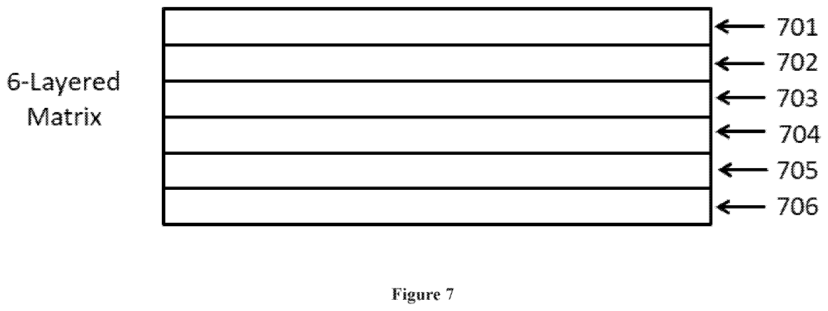

FIG. 7 illustrates a six-layer transdermal patch comprising a release liner (701), a backing film (706), and a four-layer adhesive matrix in between. The 4-layered adhesive matrix is composed of a first Drug Matrix (702, containing pramipexole), a Controlling Matrix (703), a Placebo Matrix (704) and a second Drug Matrix (705, containing pramipexole). The first Drug Matrix is attached to the release liner and the second Drug Matrix is adhered to the backing film. The release liner needs to be peeled off to expose the adhesive matrix before use.

### Examples 20 (General Manufacturing Process to Make a 4-Layered Adhesive Matrix)

The general manufacturing process to make a transdermal patch composing 4-layered adhesive matrix is described as follows.
1. Preparation of PIB Solution: Weigh and add HMW PIB and LMW PIB in a container at a weight ratio of 1:1.25. Add n-heptane 3 times the weight of PIB adhesives into the same container. Stir the mixture until homogeneous.
2. Pramipexole in PIB Solution: Weigh and add pramipexole in a container. Add mineral oil into the sample container. Homogenize the mixture to make a homogeneous mixture. Weigh and add PIB solution into the same container. Homogenize the mixture until homogenous.
3. Controlling Materials in PIB Solution: Weigh and add CSD and n-heptane into container 1. Homogenize the mixture until a homogeneous gel like mixture is obtained. Weigh and add Pemulen TR-2 and mineral oil in container 2. Homogenize the mixture until a creamy mixture is obtained. Add appropriate amounts of contents in container 1 and 2 into container 3. Weigh and add PIB solution into container 3. Homogenize the mixture until homogeneous.
4. Placebo Solution: Add mineral oil in a container. Weigh and add PIB solution into the same container. Stir the mixture until homogeneous.
5. Rasagiline in PIB Solution: Weigh and add rasagiline and mineral oil in a container. Warm the mixture at 40 °C to dissolve rasagiline. Weigh and add PIB solution into the container. Mix all ingredients to form a homogeneous solution.
6. Coating, drying and lamination of the above solutions:
   6.1 Coat Pramipexole in PIB Solution on a sheet of release liner. Dry the solution at 122 °F for 3 minutes to obtain the first Drug Matrix.
   6.2 Coat Controlling Materials in PIB Solution on the first Drug Matrix. Dry the solution at 122 °F for 10 minutes to obtain Controlling Matrix on the first Drug Matrix.
   6.3 Coat Placebo in PIB Solution on Controlling Matrix. Dry the solution at 122 °F for 3 minutes to obtain Drug Placebo Matrix on Controlling Matrix. Put the sheet aside.
   6.4 Coat Rasagiline in PIB Solution on a sheet of backing film. Dry the solution at 122 °F for 3 minutes to obtain the second Drug Matrix.
7 Lamination: Laminate the surface of the Controlling Matrix of step 6.3 with the surface of the second Drug Matrix of step 6.4 to make a sheet which is composed of release liner, a laminate of 4-adhesive matrix and backing film.
8. Cutting: Cut the sheet after lamination into patches of 5 cm² each under Carver press.

### Examples 21-23 Effect of Pemulen TR-2 Percentage in the Controlling Matrix on the Skin Delivery of Rasagiline

Three examples of rasagiline containing patches, each made of a four-layer adhesive matrix, are prepared (Table 9). The formulations of these three patches differ in the percentage of Pemulen TR-2 in the Controlling Matrix. Table 9 lists the formulation components.

TABLE 9
Formulations of Examples 21-23

| | | Example | | |
|---|---|---|---|---|
| | | 21 | 22 | 23 |
| Layer | Ingredients | Weight % | | |
| Skin Contact | CSD | 7.5 | 7.5 | 7.5 |
| Matrix | Mineral Oil | 36.4 | 36.4 | 36.4 |
| | Oppanol N-100 | 24.9 | 24.9 | 24.9 |
| | Oppanol B-10 | 31.2 | 31.2 | 31.2 |
| Controlling | CSD | 7.5 | 7.5 | 7.5 |
| Matrix | Pemulen TR-2 | 10.0 | 12.0 | 15.0 |
| | Mineral Oil | 32.5 | 31.7 | 30.5 |
| | Oppanol N-100 | 22.2 | 21.7 | 20.8 |
| | Oppanol B-10 | 27.8 | 27.1 | 26.1 |
| Placebo Matrix | Mineral Oil | 39.4 | 39.4 | 39.4 |
| | Oppanol N-100 | 26.9 | 26.9 | 26.9 |
| | Oppanol B-10 | 33.7 | 33.7 | 33.7 |
| Rasagiline | Rasagiline | 5.0 | 5.0 | 5.0 |
| Matrix | Mineral Oil | 37.4 | 37.4 | 37.4 |
| | Oppanol N-100 | 25.6 | 25.6 | 25.6 |
| | Oppanol B-10 | 32.0 | 32.0 | 32.0 |

FIG. 8 shows the skin permeation profiles. As shown, the 10% Pemulen TR-2 in the Controlling Matrix is showing the higher initial skin permeation than those having a higher percentages of Pemulen TR-2. However, there is not much difference if Pemulen TR-2 is incorporated at above 12% level.

### Examples 24-25 (to Understand if Pramipexole and Rasagiline can be Formulated in the Same Layer)

Four-layered transdermal patches are designed. Table 10 shows that Example 24 is having both pramipexole and rasagiline in the second Drug Matrix. Example 25 is having pramipexole in the first Drug Matrix and rasagiline in the second Drug Matrix. Manufacturing process is described in Example 18.

TABLE 10
Formulations of Examples 24-25

| | | Example | |
|---|---|---|---|
| | | 24 | 25 |
| Layer | Ingredients | Weight % | |
| The first | Pramipexole | | 6 |
| Drug Matrix | CSD | 7.5 | |
| | Pemulen TR-2 | | 1.5 |
| | Mineral Oil | 36.4 | 36.4 |
| | Oppanol N-100 | 24.9 | 24.9 |
| | Oppanol B-10 | 31.2 | 31.2 |
| Controlling | CSD | 7.5 | 7.5 |
| Matrix | Pemulen TR-2 | 12.0 | 12.0 |
| | Mineral Oil | 31.7 | 31.7 |
| | Oppanol N-100 | 21.7 | 21.7 |
| | Oppanol B-10 | 27.1 | 27.1 |
| Placebo Matrix | Mineral Oil | 39.4 | 39.4 |
| | Oppanol N-100 | 26.9 | 26.9 |
| | Oppanol B-10 | 33.7 | 33.7 |
| The second | Pramipexole | 3.5 | |
| Drug Matrix | Rasagiline | 2.0 | 2.0 |
| | Mineral Oil | 37.2 | 38.6 |
| | Oppanol N-100 | 25.4 | 26.4 |
| | Oppanol B-10 | 31.8 | 33.0 |

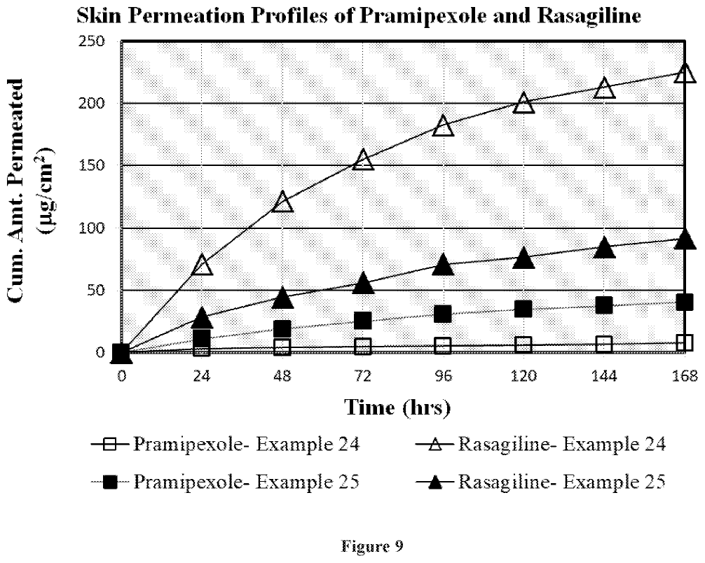

For Example 24, both pramipexole and rasagiline have to diffuse through the controlling matrix before released from the matrix and permeated through skin. FIG. 9 indicated Pramipexole and rasagiline show the amount of skin permeation at 0.007 mg/cm² and 0.225 mg/cm², respectively after 7 days. However, for Example 25, different from rasagiline, pramipexole does not need to diffuse through the controlling matrix. Pramipexole and rasagiline show the amount of skin permeation at 0.04 mg/cm² and 0.09 mg/cm², respectively after 7 days. The results indicate that the release of pramipexole from the adhesive matrix is regulated by the controlling layer. In addition, the release of pramipexole and rasagiline is competing with each other, i.e., with a higher skin permeation of pramipexole the skin permeation of rasagiline will be delayed.

### Examples 26-27 (Effect of Thickness of Controlling Matrix on Skin Permeation of Pramipexole and Rasagiline)

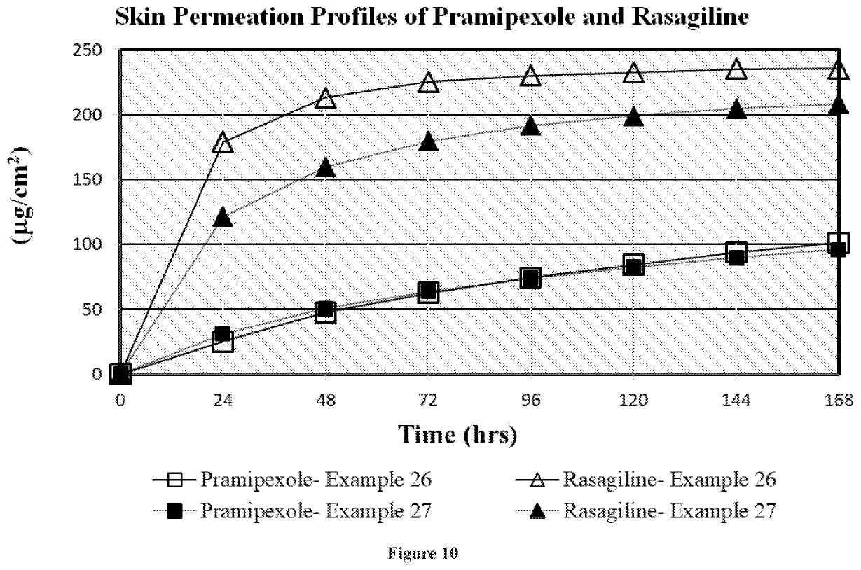

Table 11 shows the formulation of the present study. Example 26 and Example 27 differ in the coating thickness of the Controlling Matrix, of which the thickness in Example 26 and Example 27 are 150 μm and 300 μm, respectively before drying. FIG. 10 shows the skin permeation profiles of pramipexole and rasagiline. Example 26 with a thinner coating of the Controlling Matrix, the skin permeation of rasagiline is faster than that with a thicker coating as seen in Example 27. However, since pramipexole does not need to go through Controlling Matrix, its skin permeation profiles are not impacted by the coating thickness of Controlling Matrix.

TABLE 11
Formulations of Examples 26-27

| | | Formulation |
|---|---|---|
| Layer | Ingredients | Weight % |
| The first | Pramipexole | 6 |
| Drug Matrix | Pemulen TR-2 | 1.5 |
| | Mineral Oil | 36.4 |
| | Oppanol N-100 | 24.9 |
| | Oppanol B-10 | 31.2 |
| Placebo Matrix | Mineral Oil | 39.4 |
| | Oppanol N-100 | 26.9 |
| | Oppanol B-10 | 33.7 |
| Controlling | CSD | 12.0 |
| Matrix | Pemulen TR-2 | 3.0 |
| | Mineral Oil | 33.5 |
| | Oppanol N-100 | 22.9 |
| | Oppanol B-10 | 28.6 |
| The second | Rasagiline | 2.5 |
| Drug Matrix | Mineral Oil | 38.4 |
| | Oppanol N-100 | 26.3 |
| | Oppanol B-10 | 32.8 |

### Example 28 (a Multi-Layered Transdermal Patch and the Skin Permeation Results)

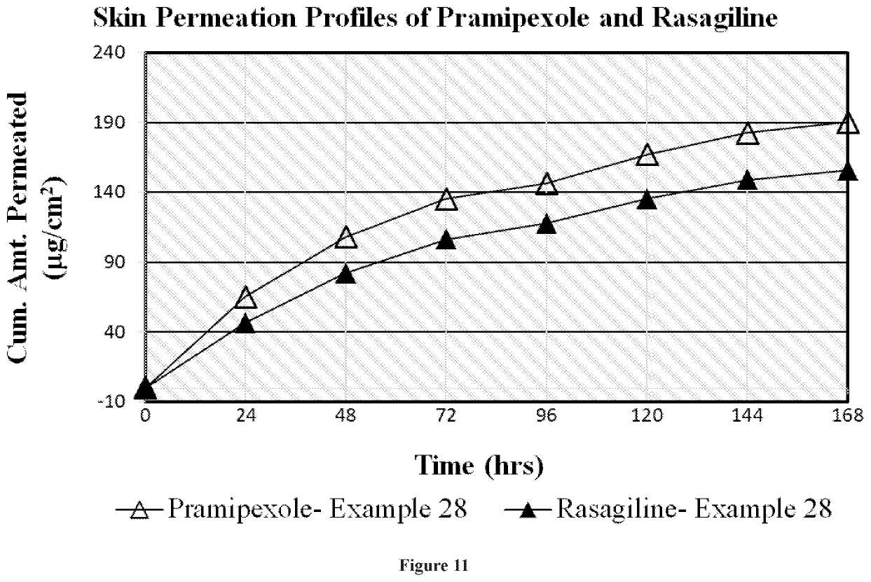

Table 12 shows the formulation and FIG. 11 shows the skin permeation results of Example 28.

TABLE 12
Formulations of Example 28

| | | Formulation |
|---|---|---|
| Layer | Ingredients | Weight % |
| The first | Pramipexole | 9 |
| Drug Matrix | Mineral Oil | 35.9 |
| | Oppanol N-100 | 24.5 |
| | Oppanol B-10 | 30.7 |
| Controlling | CSD | 7.5 |
| Matrix | Pemulen TR-2 | 12.0 |
| | Mineral Oil | 31.7 |
| | Oppanol N-100 | 21.7 |
| | Oppanol B-10 | 27.1 |
| Placebo Matrix | Mineral Oil | 39.4 |
| | Oppanol N-100 | 26.9 |
| | Oppanol B-10 | 33.7 |
| The second | Rasagiline | 1.5 |
| Drug Matrix | Mineral Oil | 38.8 |
| | Oppanol N-100 | 26.5 |
| | Oppanol B-10 | 33.2 |

FIG. 11 indicates that pramipexole and rasagiline are permeated through skin samples at controlled linear delivery profiles during a 7-day period of the study.

The present application discloses a transdermal patch which is composed of release liner, a multi-layered adhesive matrix and backing film. The multi-layered adhesive matrix is a laminate of the first Drug Matrix which contains pramipexole in PIB adhesive matrix, Controlling Matrix which is composed of Pemulen TR-2, a tackifier, and colloidal silicone dioxide, a filler in PIB adhesive matrix, Placebo Matrix, and a second Drug Matrix which contains rasagiline in PIB adhesive matrix. Based on the above study results, the patch of the present invention can release pramipexole and rasagiline continuously at controlled rates for a long duration.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A transdermal patch to steadily deliver two pharmaceutical drugs, wherein the transdermal patch comprises a release liner, a multi-layered adhesive matrix and a backing film; wherein the two pharmaceutical drugs are pramipexole and rasagiline; wherein the multi-layered adhesive matrix laminate comprises:
(a) a release liner;
(b) a first drug matrix comprising pramipexole free base,
(c) a controlling matrix comprising Acrylates C10-30 Alkyl Acrylate Crosspolymer,
(d) a placebo matrix;
(e) a second drug matrix comprising rasagiline free base; and
(f) a backing film,
wherein the first drug matrix, the controlling matrix and the second drug matrix are laminated between the release liner and the backing film.

2. The transdermal patch according to claim 1, wherein the Acrylates C10-30 Alkyl Acrylate Crosspolymer in the controlling matrix is incorporated in an amount of 2-12 wt %.

3. The transdermal patch according to claim 1, wherein the transdermal patch delivers pramipexole at a skin permeation rate of 0.35-3.5 μg/cm² per hour.

4. The transdermal patch according to claim 1, wherein the transdermal patch delivers rasagiline at a skin permeation rate of 0.2-3.6 μg/cm² per hour.

5. The transdermal patch according to claim 1, wherein the transdermal patch can steadily deliver pramipexole and rasagiline for 7 days.

6. A method of manufacturing a transdermal patch comprising rasagiline, the method comprising:
(a) Preparation of polyisobutylene (PIB) Solution: dissolve a high molecular weight PIB and a low molecular weight PIB in N-Heptane;
(b) Rasagiline in PIB Solution: dissolve rasagiline in mineral oil at 40 °C, then added PIB solution into the mixture;
(c) Coating, Drying and Lamination: the rasagiline in PIB solution is cast on a sheet of release liner, and dried in an oven to evaporate n-heptane at pre-determined drying temperature and drying time; then the dried film is laminated with a sheet of backing film to form a 3-layered laminate.

7. The method of manufacturing a transdermal patch comprising rasagiline in claim 6, wherein the high molecular weight PIB is HMW PIB-Oppanol N-100, and the low molecular weight PIB is LMW PIB-Oppanol B-10.

8. The method of manufacturing a transdermal patch comprising rasagiline in claim 6, wherein the weight ratio of the high molecular weight PIB, the low molecular weight PIB and the N-Heptane is 1:1.25:6.75.

9. The method of manufacturing a transdermal patch comprising rasagiline in claim 6, wherein the pre-determined drying temperature and drying time is 113-149 °F for 3-5 minutes.

10. The method of manufacturing a transdermal patch comprising rasagiline in claim 9, wherein the pre-determined drying temperature and drying time is 122 °F for 3 minutes.
